(19) United States
(12) Patent Application Publication
(10) Pub. No.: US 2015/0366621 A1
(43) Pub. Date: Dec. 24, 2015

(54) MEDICAL INSTRUMENT HOLDING APPARATUS

(71) Applicant: Hiwin Technologies Corp., Taichung (TW)

(72) Inventor: Ren Jeng WANG, Taichung (TW)

(21) Appl. No.: 14/642,813

(22) Filed: Mar. 10, 2015

(30) Foreign Application Priority Data
Jun. 23, 2014 (TW) 103121557

(51) Int. Cl. A61B 19/00

(52) U.S. Cl. CPC A61B 19/201

(57) ABSTRACT

A medical instrument holding apparatus includes a supporting mechanism having an extension which includes a bore, a medical instrument, and an attachment device includes a shaft slidably engaged in the bore of the extension, and the shaft includes one end portion secured to the medical instrument, and the attachment device includes a handle provided on the shaft, and the shaft is adjustable relative to the extension of the supporting mechanism to a selected position. The extension includes a slot communicating with the bore of the extension for forming two separated flaps, and includes a fastener engaged with the flaps for forcing the flaps to engage with the shaft and to detachably and adjustably secure the shaft to the extension of the supporting mechanism.

### BACKGROUND OF THE INVENTION

1. Field of the Invention

The present invention relates to a medical instrument holding apparatus, and more particularly to a medical instrument holding apparatus including an improved or simplified structure or configuration that may be easily and quickly made or manufactured by the manufacturers and that may be easily used to conduct the operation on the patient for allowing the medical surgery or medical operation to be easily conducted or actuated by the users or operators.

2. Description of the Prior Art

Typical medical instruments, such as rigidoscopes, laparoscopes, endoscopes, trocars, treating instruments, or the other medical instruments or the like have been developed and widely provided and used for conducting or operating an operation on a patient, and are typically grasped and held by the hands of the operators, such as the doctors, and it will be difficult for the operators or the doctors to hold the medical instruments and to conduct or operate the operation on the patient.

Normally, the medical instruments are grasped and held by one of the operators or the doctors, and the other operator or doctor will stand beside the medical instruments holding operator and will both standing near the patient at the same time, in a tiny or narrowed operation room.

For allowing the medical instruments to be held in place without being grasped by the operators or the doctors, a scope-holder has been disclosed and used to hold the medical instruments in place, for inserting the medical instruments into the abdominal cavity of a patient.

For example, U.S. Pat. No. 5,697,939 to Kubota et al. discloses one of the typical medical instrument holding apparatuses comprising a plurality of arms sections and holding sections for holding and grasping the treating instrument or medical instrument and for engaging or inserting the treating or medical instruments into the abdominal cavity of the patient.

However, the typical medical instrument holding apparatuses comprise a rather complicated structure or configuration having a number of parts or elements that may not be easily and quickly made or manufactured and that may not be easily used to conduct the operation on the patient.

U.S. Pat. No. 8,287,522 to Moses et al. discloses another typical medical instrument or device comprising a plurality of arms sections and holding sections for holding and grasping the treating instrument or medical instrument.

However, the typical medical instrument or device may not be easily operated or actuated by the users or operators.

The present invention has arisen to mitigate and/or obviate the afore-described disadvantages of the conventional medical instrument holding apparatuses.

### SUMMARY OF THE INVENTION

The primary objective of the present invention is to provide a medical instrument holding apparatus including an improved or simplified structure or configuration that may be easily and quickly made or manufactured by the manufacturers and that may be easily used to conduct the operation on the patient for allowing the medical surgery or medical operation to be easily conducted or actuated by the users or operators.

In accordance with one aspect of the invention, there is provided a medical instrument holding apparatus comprising a supporting mechanism including an extension extended therefrom, the extension including a bore formed therein, a medical instrument, and an attachment device including a shaft slidably engaged in the bore of the extension, and the shaft including a first end portion secured to the medical instrument, and the attachment device including a handle provided on the shaft, and the shaft being adjustable relative to the extension of the supporting mechanism to a selected position.

The extension includes a slot formed therein and communicating with the bore of the extension for forming two separated flaps, and includes a fastener engaged with the flaps for forcing the flaps to engage with the shaft.

The medical instrument includes a seat secured to the first end portion of the shaft. The seat includes an opening formed therein for engaging with the first end portion of the shaft. The seat includes a slit formed therein and communicating with the opening of the seat for forming two ears, and a fastener engaged with the ears.

The attachment device includes a button provided on the handle for operating or actuating or moving the medical instrument relative to the supporting mechanism.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a partial exploded view illustrating the operation of a medical instrument holding apparatus in accordance with the present invention;

FIG. 2 is a perspective view of the medical instrument holding apparatus;

FIG. 3 is a side plan schematic view of the medical instrument holding apparatus;

FIG. 4 is another side plan schematic view similar to FIG. 3, illustrating the operation of the medical instrument holding apparatus;

FIG. 5 is a partial side plan schematic view of the medical instrument holding apparatus, in which a portion of the medical instrument holding apparatus has been cut off for showing the inner structure of the medical instrument holding apparatus; and

FIGS. 6 and 7 are cross sectional views of the medical instrument holding apparatus, taken along lines 6-6 and 7-7 respectively.

### DETAILED DESCRIPTION OF THE PREFERRED EMBODIMENT

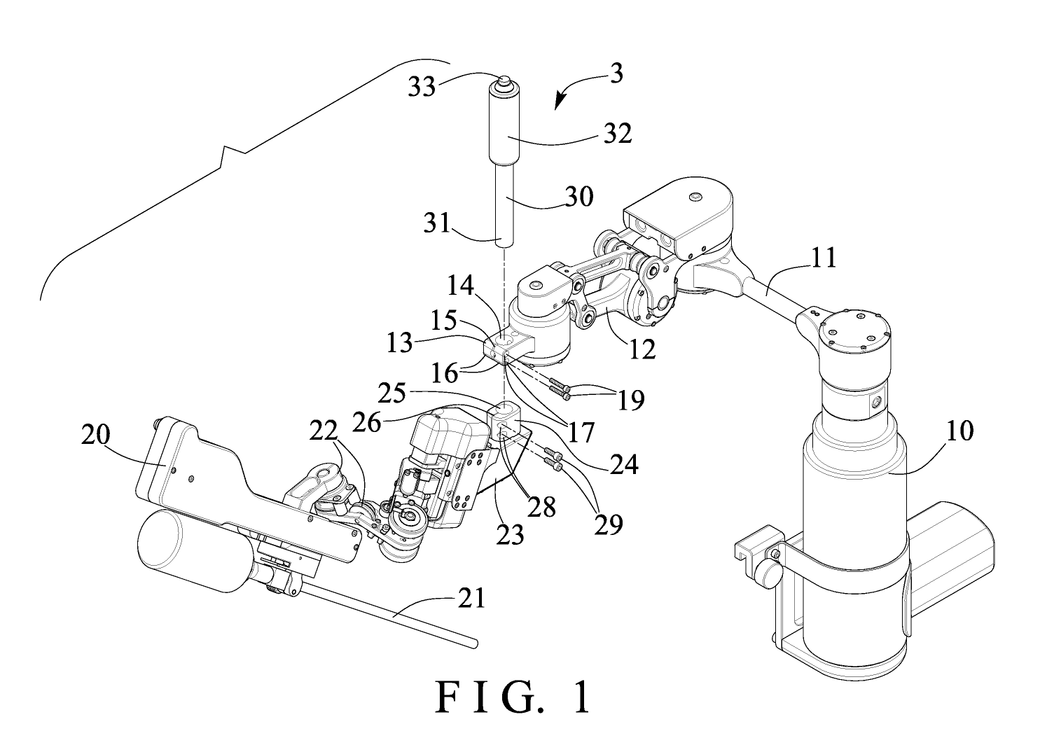

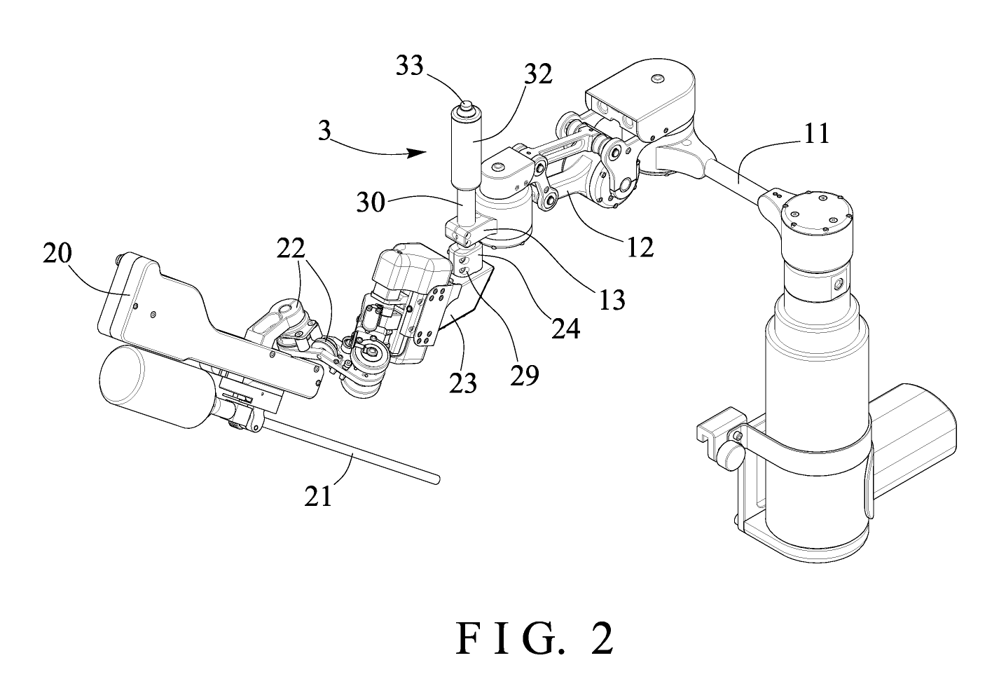

Referring to the drawings, and initially to FIGS. 1-4, a medical instrument holding apparatus in accordance with the present invention comprises a stay or supporting device or mechanism 10 including one or more arms 11 and levers 12 pivotally or rotatably or slidably connected or coupled together for allowing the arms 11 and the levers 12 to be pivoted or rotated or moved relative to each other. The connecting or coupling of the arms 11 and the levers 12 of the supporting mechanism 10 is typical and is not related to the present invention and will not be described in further details. The supporting mechanism 10 includes a stud or extension 13 extended outwardly therefrom, such as extended outwardly from either of the arms 11 or the levers 12.

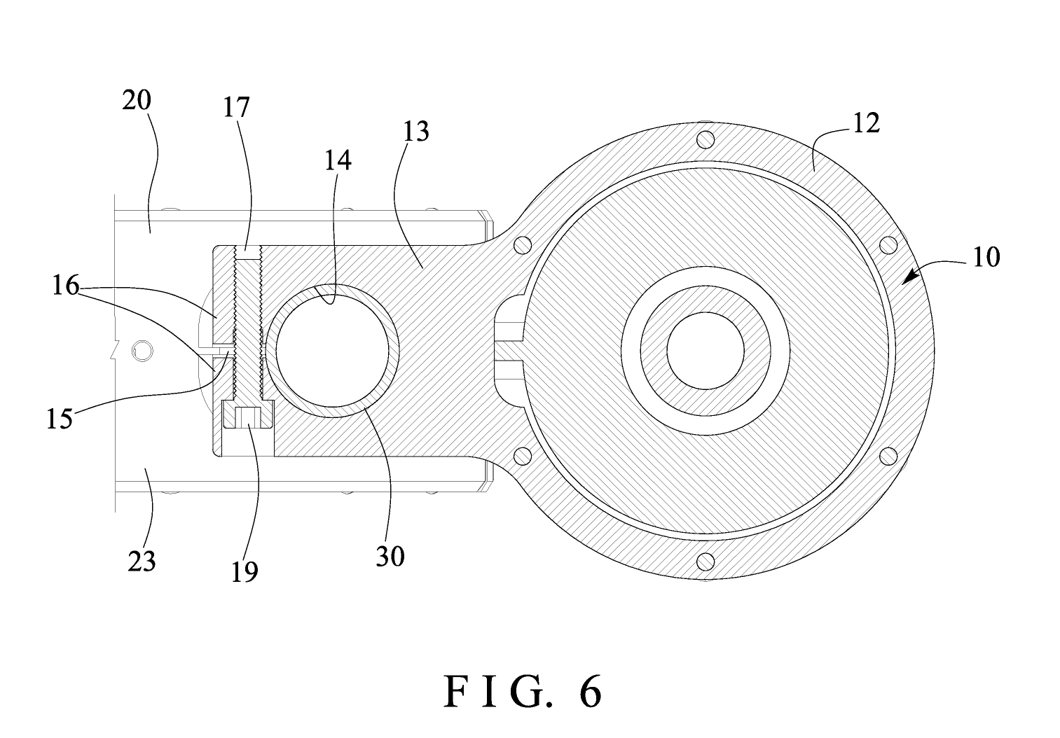

As also shown in FIGS. 1 and 5-6, the extension 13 of the supporting mechanism 10 includes a hole or bore 14 formed therein, and includes a groove or slot 15 formed therein and intersected or communicating with the bore 14 of the extension 13 for forming or defining two separated ears or flaps 16 (FIGS. 1, 6) and arranged for allowing the flaps 16 to be moved or forced toward each other, and further includes one or more (such as two) screw holes or orifices 17 formed therein and through the flaps 16 for receiving or engaging with locks or fasteners 19 that may be engaged with the flaps 16 for selectively forcing the flaps 16 to move toward or away from each other.

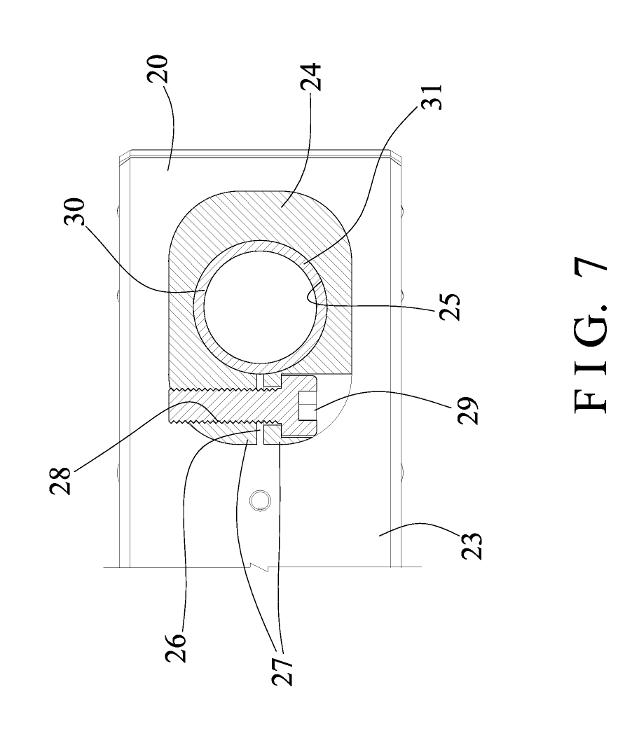

The medical instrument holding apparatus in accordance with the present invention is provided for supporting or sustaining or engaging with a medical instrument 20, such as rigidoscope, laparoscope, endoscope, trocar, treating instrument, or the other medical instruments or the like, in which the medical instrument 20 normally includes an insertion tube 21 extended therefrom for inserting or engaging into the abdominal cavity of a patient (not illustrated), the insertion tube 21 and the medical instrument 20 are also typical and are not related to the present invention and will not be described in further details. The medical instrument 20 includes one or more bars 22 and rods 23 pivotally or rotatably or slidably connected or coupled together for allowing the bars 22 and the rods 23 to be pivoted or rotated or moved relative to each other. The medical instrument 20 further includes a base or seat 24 having a hole or opening 25 formed therein, and includes a groove or slit 26 formed therein and intersected or communicating with the opening 25 of the seat 24 for forming or defining two separated flaps or ears 27 (FIGS. 1, 7) and arranged for allowing the ears 27 to be moved or forced toward each other, and further includes one or more (such as two) screw holes or apertures 28 formed therein and through the ears 27 for receiving or engaging with locks or fasteners 29 that may be engaged with the ears 27 for selectively forcing the ears 27 to move toward or away from each other. The medical instrument holding apparatus in accordance with the present invention further includes a connecting or coupling or attachment device 3 for coupling or connecting the supporting mechanism 10 and the medical instrument 20 together.

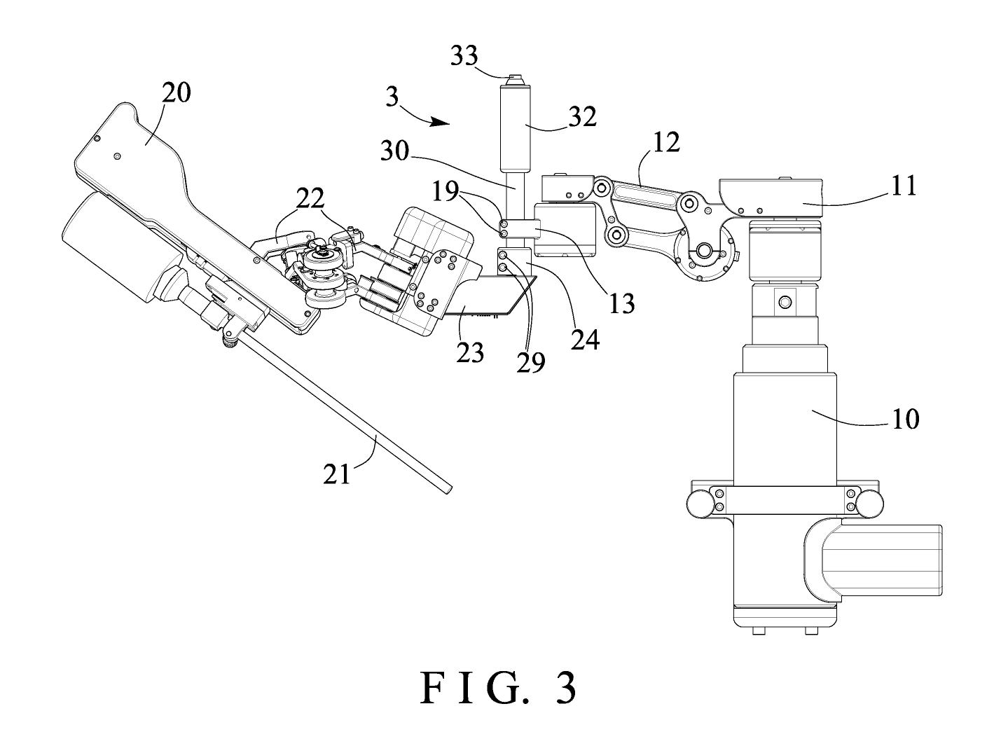

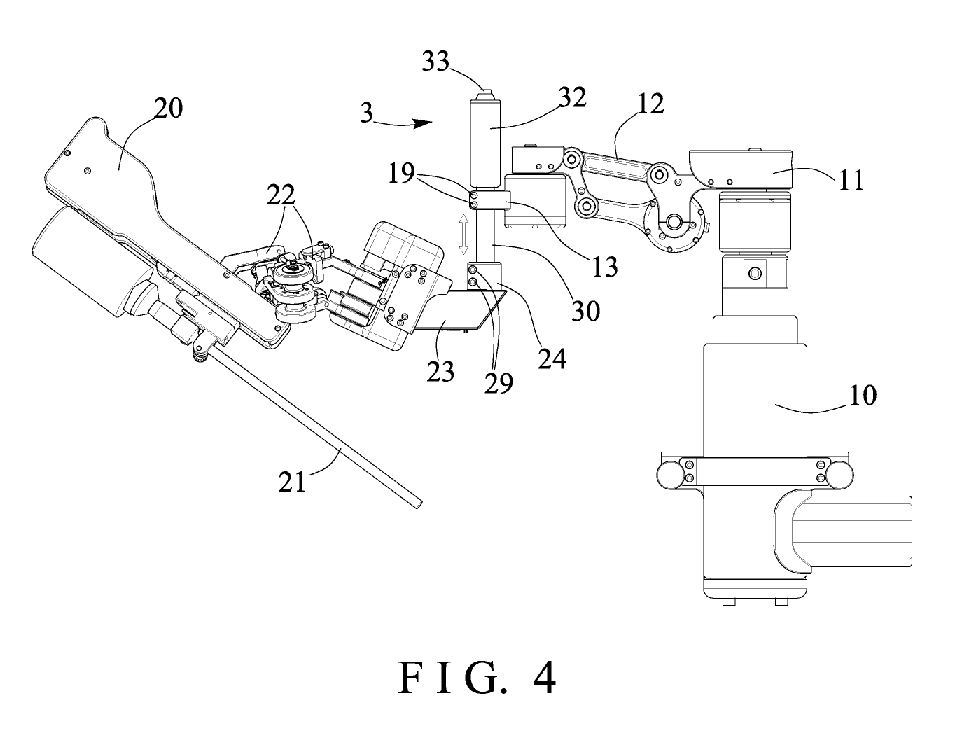

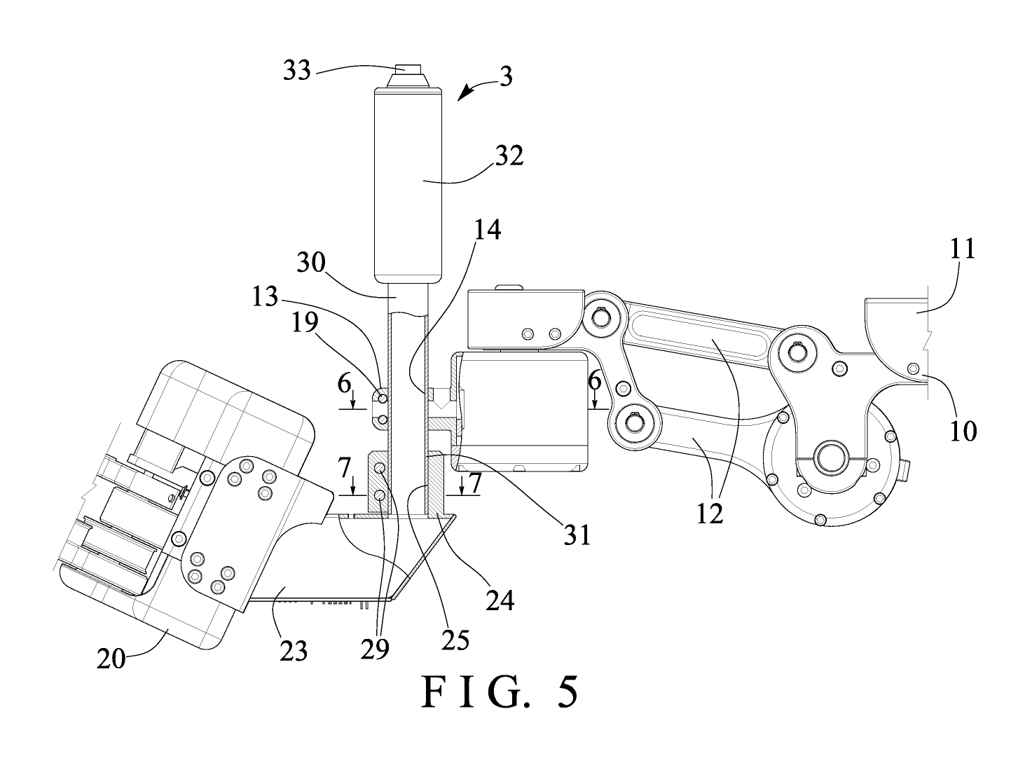

For example, the attachment device 3 includes a spindle or shaft 30 slidably received or engaged in the bore 14 of the extension 13 of the supporting mechanism 10 (FIGS. 3, 4), and the shaft 30 includes one or first or free end portion 31 engaged into the opening 25 of the seat 24 of either of the bars 22 or the rods 23 of the medical instrument 20 (FIGS. 5, 7), and detachably or removably attached or secured to the seat 24 of the medical instrument 20 with the fasteners 29, and the attachment device 3 further includes a hand grip or handle 32 formed or provided on the shaft 30 for being held or grasped by the users who may move the medical instrument 20 and/or the arms 11 and the levers 12 relative to the supporting mechanism 10. The shaft 30 is slidably or adjustably secured to the extension 13 of the supporting mechanism 10 at selected positions (FIGS. 3, 4) with the fasteners 19 which may force the flaps 16 to move to engage with the shaft 30.

The attachment device 3 may further include a knob or button 33 formed or provided on the handle 32 for depressed or actuated by the users who may use the button 33 to control the actuation or operation of the medical instrument 20, for example, but the control or the actuation or the operation of the medical instrument 20 is not related to the present invention and will not be described in further details. In operation, as shown in FIGS. 1-5, the shaft 30 may be slid or adjusted relative to the extension 13 of the supporting mechanism 10 to the required or selected positions with the fasteners 19, and the user may hold or grasp the handle 32 to move the medical instrument 20 and/or the arms 11 and the levers 12 relative to the supporting mechanism 10 to the required or selected positions, and the user may depress or actuate the button 33 to control or actuate or operate the medical instrument 20.

Accordingly, the medical instrument holding apparatus in accordance with the present invention includes an improved or simplified structure or configuration that may be easily and quickly made or manufactured by the manufacturers and that may be easily used to conduct the operation on the patient for allowing the medical surgery or medical operation to be easily conducted or actuated by the users or operators.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A medical instrument holding apparatus comprising:
a supporting mechanism including an extension extended therefrom, said extension including a bore formed therein,
a medical instrument, and
an attachment device including a shaft slidably engaged in said bore of said extension, and said shaft including a first end portion secured to said medical instrument, and said attachment device including a handle provided on said shaft, and said shaft being adjustable relative to said extension of said supporting mechanism to a selected position.

2. The medical instrument holding apparatus as claimed in claim 1, wherein said extension includes a slot communicating with said bore of said extension for forming two flaps, and a fastener engaged with said flaps for forcing said flaps to engage with said shaft.

3. The medical instrument holding apparatus as claimed in claim 1, wherein said medical instrument includes a seat having an opening formed therein for engaging with said first end portion of said shaft.

4. The medical instrument holding apparatus as claimed in claim 3, wherein said seat includes a slit formed therein and communicating with said opening of said seat for forming two ears, and a fastener engaged with said ears.

5. The medical instrument holding apparatus as claimed in claim 1, wherein said attachment device includes a button provided on said handle.
